(19) United States
(12) Patent Application Publication
(10) Pub. No.: US 2002/0194031 A1
(43) Pub. Date: Dec. 19, 2002

(54) Method for acquiring and evaluating data during the admission of a patient for operation

(76) Inventors: Norman Bitterlich, Chemnitz (DE); Thomas Loeser, Leipzig (DE)

Correspondence Address: JORDAN AND HAMBURG LLP, 122 EAST 42ND STREET, SUITE 4000, NEW YORK, NY, 10168, US

(21) Appl. No.: 10/018,079

(22) PCT Filed: May 10, 2001
(86) PCT No.: PCT/EP01/05369

(30) Foreign Application Priority Data
May 12, 2000 (DE) 100 23159.4

(51) Int. Cl. G06F 17/60; G06F 19/00; G01N 33/48; G01N 33/50

(52) U.S. Cl. 705/3; 702/19

(57) ABSTRACT

The aim of the invention is to develop a method for the acquisition and evaluation of data during the admission of an operation patient, enabling the doctor to objectively justify a decision when classifying the operation of patient in one of the risk groups. To this end, the invention provides that the patient's data is acquired electronically. All patient admission inquiries are made automatically using software, via an interactive data acquisition unit. A risk evaluation is provided throughout the data acquisition process, based on the current status of the data. During the data acquisition process, a list of still urgently required entries is provided with each risk evaluation and as yet unanswered questions are acknowledged as such in order to register the fulfillment of the duty of care in recording the data.

The invention relates to a method for acquiring and evaluating data during the admission of a patient for surgery. Any operation represents and an enormous intervention in the organism of the patient concerned. In spite of medical art and comprehensive medical and technical support, complications basically cannot be excluded. It is therefore an important prerequisite for surgery to determine and evaluate the general state of the health of the patient, in order to identify possible complications and to make preparations for appropriate measures. For this purpose, each patient is assigned to one of several specified risk groups. For personnel and organizational reasons, this evaluation furthermore enters into the surgical planning, in order to avoid an accumulation of high-risk operations.

In general, the period between the admission of a patient and the surgery is so short, that comprehensive clinical pre-admission testing for determining the risk group cannot be carried out. Aside from a few laboratory values, the decision is therefore based on a knowledge of the case history and of the long-term medication, obtained by asking the patient, provided that the latter is in a position to provide such information. From these data, the admitting physician must make a decision concerning the fitness of the patient for surgery. For this, the physician's own practical experience plays a dominant role since, with the present state of the art, an objectivized decision aid is not available.

It is therefore an object of the invention to develop a method for acquiring and evaluating data during the admission of a patient for surgery, with which it is possible to make an objectivized decision aid for the classification of the surgical patient into risk groups available to the physician during the admission of the patient for surgery.

Pursuant to the invention, this objective is accomplished owing to the fact that the patient data is acquired electronically, all inquiries concerning the admission of the patient being made automatically over a programmed, interactive data acquisition unit, so that

a routine sequence of basic questions is maintained,

the input is checked for compatibility and plausibility,

the completeness of the information is checked in relation to risk evaluation,

optionally, additional necessary information is obtained,

the absence of information with regard to the consequences for the evaluating unit is estimated and

all admission activities are recorded,

that, at any time of the data acquisition, the risk is evaluated from the actual state of the information, that, in the course of the data acquisition, with each risk evaluation, a list is presented of further urgently necessary entries and that unanswered questions are acknowledged as such, in order to record that the due diligence obligation during the data acquisition has been fulfilled.

It is advantageous that the risk is evaluated at every point in the data acquisition from the actual state of the information so that

a.) each input value of the data acquisition is given a value between 0 and 1 in regard to its effect on the surgical risk, a 0 being assigned if a risk-increasing effect on the course of the surgery is not to be expected from concrete information, and the value of 1 being assigned if, on the base of experience, a dramatic surgical complication cannot be excluded for the concrete information provided,

b.) each input field in an input device is occupied by a standard number for the risk assignment to a risk group

1=none

2=slight

3=moderate

4=serious and

5=dramatic surgical complications

the standard numbers being modified by authorized users and adapted by real reference data;

c.) the input fields are grouped according to their contents and, for the individual groups, the respective group affiliation value μ_{G} is determined from the risk group numbers using the Fuzzy Set Theory;

d.) the individual group affiliation values are combined into a total affiliation μ* over a rule-based fuzzy system and

e.) the risk evaluation is obtained from the overall affiliation μ*, in that this value, between 1 and 5, is transformed by rounding off into a whole number between 1 and 5 of the risk group:

R*_{PAT}=Round(μ*)

In the following, the invention is described in greater detail by means of an example. The starting point is the electronic acquisition of patient data, all inquiries in relation to the admission of the patient being made automatically using a programmed, interactive data acquisition unit, so that

a routine sequence of basic questions is maintained,

the input is checked for compatibility and plausibility,

the completeness of the information is checked in relation to risk evaluation,

optionally, additional necessary information is obtained,

the absence of information with regard to the consequences for the evaluating unit is estimated and

all admission activities are recorded,

At any time of the data acquisition, the risk is evaluated from the actual state of the information and a list is provided of further urgently necessary entries. Unanswered questions must be acknowledged as such, in order to record that the due diligence obligation during the data acquisition has been fulfilled. The result of the risk evaluation is also to be acknowledged. At the same time, possibilities for change on the basis of a deviating subjective evaluation are explicitly offered. The evaluation of the risk is demonstrated by means of an example of a data set. Each input value of the data acquired contains a number between 0 and 1, which is indicative of its effect on the surgical risk. The system is configured in a standard way. The input field <<Age>> contains the following risk groups:

| Male | | Risk | Female | | Risk |
|---|---|---|---|---|---|
| Risk Group | | Number | Risk Group | | Number |
| Group 1 | up to 30 years | 0.0 | Group 1 | up to 30 years | 0.0 |
| Group 2 | 31 to 65 years | 0.2 | Group 2 | 31 to 60 years | 0.2 |
| Group 3 | 66 to 80 years | 0.4 | Group 3 | 61 to 75 years | 0.4 |
| Group 4 | older than 81 | 0.5 | Group 4 | older than 76 | 0.5 |
| | years | | | years | |
| Group 5 | — | | Group 5 | — | |

The body mass index (BMI) is determined from the height and weight and is assigned to risk groups in the following way:

| Risk | | Risk |
|---|---|---|
| Group | | Number |
| Group 1: | BMI of l5 to 30 | 0.0 |
| Group 2: | BMI of less than 15 or BMI between 30 and 45 | 0.3 |
| Group 3: | BMI greater than 45 | 0.6 |
| Groups 4/5 | — | |

The person in charge has started the system and, in accordance with his user identification, has entered his name.

Risk Evaluation:

The patient goes to Admissions for a surgical intervention, which has been planned a long time. He presents his chip card for acquiring the personal data. This card is inserted in a reader. With the actuation of the input field, Chip Card, the reading process is initiated and the personal data is imported. The height and weight of the patient are entered.

Data Acquisition System for Surgical
Patients - Personal Data
Risk Group 2.00

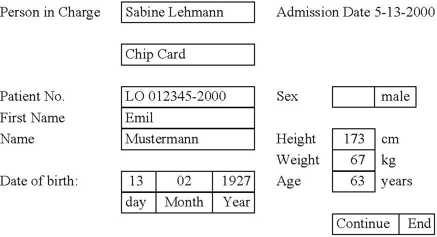

The risk coefficients for the age (63 years) and for BMI (22) are 0.4 and 0.0 respectively. With the Fuzzy Set Theory method, namely the algebraic product of the negated risk numbers, the group affiliation value μ_{G} is 1−(1−0.4)×(1 −0.0)=0.4. The risk group is 2.0.

As a result of pre-admission testing, the laboratory values are available for a planned intervention. By activating the input field <<DFÜ>>, the available data are polled and automatically read in. The input fields, <<Case History>> and <<Medication>>, lead to sub-menus, the details of which are filled in sequentially by asking the patient. For each group, a risk value, corresponding to the underlying characteristic values, is calculated and indicated automatically.

The group affiliation values μ_{G}, personal data, case history and medication are combined into an overall affiliation μ* with a rule-based fuzzy system, which is parameterized in the following way:

each of the input quantities is fuzzified to the value range {0:6} by means of five triangular functions, the peaks of which are defined by the points (1.0; 2.0; 3.0; 4.0; 5.0) and which add up to 1,

the initial quantities, as singletons, are defined with the values (1.0; 2.0; 3.0; 4.0; 5.0),

for the i^{th} affiliation function of the first input quantity, the J^{th} affiliation function of the second input quantity and the k^{th} affiliation function of the third input quantity, min {5; i+j+k−2} is linked with the output singleton of the number.

the Max-Max method is used for the inference

the key point method is used to defuzzify.

The value of 3.98 is obtained as the value for the overall affiliation μ*.

Data Acquisition System for Surgical
Patients - Personal Data
Risk Group 3.98

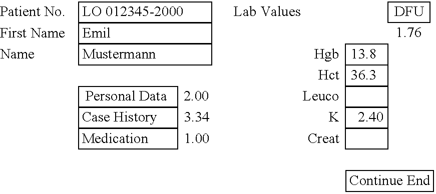

All input windows were answered, the input was concluded and the information was printed as a patient sheet.

The risk is evaluated from the overall affiliation μ*, in that this value, which lies between 1 and 5, is transformed by sounding off to a whole number between 1 and 5 of the risk groups:

R*_{PAT}=Round(μ*)

In the present case, R*=4.

The risk group can be determined at any time on the basis of the values actually entered. Any input values, not set, receive the risk affiliation of 0. Each additional input, at most, increases the risk group, so that at any time, even when the data input is still incomplete, a reliable estimate of the risk group can be made. The input, which is expected to lead to the greatest increase in the risk group, is always determined by internal simulation calculation. The input can be terminated when the highest risk group is reached or when further inputs cannot bring about an increase. Nevertheless, the input can be continued to complete the patient data.

A problem, which is currently solved unsatisfactorily because the solution is completely subjective, is solved at an entirely new level of quality with the invention. A high quality and safety in patient care is achieved with the result and associated with a reduction in routine tasks in favor of an expertly influenced control. Sources of error from the previously customary data transfer are minimized. Personnel costs are reduced because subsequent manual data transfers are avoided. Because of the increased reliability of the information, surgical measures can be planned and prepared in a more target-oriented manner, so that treatment costs are reduced. Finally, the method contributes to reducing complications. This has a direct effect on the success of the healing and the quality of life of the patient.

1. A method for acquiring and evaluating data during the admission of a patient for surgery, wherein the patient data is acquired electronically, all inquiries concerning the admission of the patient being made automatically over a programmed, interactive data acquisition unit, so that
a routine sequence of basic questions is maintained,
the input is checked for compatibility and plausibility,
the completeness of the information is checked in relation to risk evaluation,
optionally, additional necessary information is obtained,
the absence of information with regard to the consequences for the evaluating unit is estimated and
all admission activities are recorded,
that, at any time of the data acquisition, and the risk is evaluated from the actual state of the information, that, in the course of the data acquisition, with each risk evaluation, a list is presented of further urgently necessary entries and that unanswered questions are acknowledged as such, in order to record that the due diligence obligation during the data acquisition has been fulfilled.

2. The method of claim 1, wherein the risk is evaluated at every point in the data acquisition from the actual state of the information so that the
a.) each input value of the data acquisition is given a value between 0 and 1 in regard to its effect on the surgical risk, a 0 being assigned if a risk-increasing effect on the course of the surgery is not to be expected from concrete information, and the value of 1 being assigned if, on the base of experience, the dramatic surgical complications cannot be excluded for the concrete information provided,
b.) each input field in an input device is occupied by a standard number the risk assignment to a risk group
1=none
2=slight
3=moderate
4=serious and
5=dramatic surgical complications
the standard number is being modified by authorized users and adapted by real reference data;
c.) the input fields are grouped according to their contents and, for the individual groups, the respective group affiliation with μ_{G} from the risk group numbers using the Fuzzy Set Theory;
d.) the individual group affiliation values are combined into a total affiliation μ* over a rule based fuzzy system and
e.) The risk evaluation is obtained from the affiliation μ*, in that this value, between 1 and 5, is transformed by rounding off into a whole number between 1 and 5 of the risk group:
R*_{PAT}=Round(μ*)
